(19) United States
(12) Patent Application Publication
(10) Pub. No.: US 2008/0147125 A1
(43) Pub. Date: Jun. 19, 2008

(54) Active Settling Plate and Method of Use

(76) Inventors: Dennis Colleran, North Attleboro, MA (US); James Spitler, Plano, TX (US)

Correspondence Address: CARR LLP (IST), 670 FOUNDERS SQUARE, 900 JACKSON STREET, DALLAS, TX, 75202, US

(21) Appl. No.: 11/954,921

(22) Filed: Dec. 12, 2007

Related U.S. Application Data
(60) Provisional application No. 60/869,577, filed on Dec. 12, 2006.

(51) Int. Cl. A61B 17/58

(52) U.S. Cl. 606/280; 606/102

(57) ABSTRACT

Disclosed are certain embodiments of a bone plate system which use a first plate member with a plurality of bone screw holes and a receiving segment with a longitudinal portal. A second plate member may have a plurality of bone screw holes and a slider segment dimensioned to fit within the longitudinal portal of the first plate member. A first and second compression biasing members may extend generally parallel to the first and second plate members and may be laterally spaced apart from the receiving segment and the slider segment. The first and second compression biasing members may be integral with the first and second plate members so as to form a unitary member.

### CROSS REFERENCE TO RELATED APPLICATIONS

This application claims priority to U.S. Provisional Patent Application No. 60/869,577, filed Dec. 12, 2006, and entitled ACTIVE SETTLING PLATE AND METHOD OF USE, which is hereby incorporated by reference.

### TECHNICAL FIELD

The invention relates generally to instruments and methods for orthopedic surgery and, more particularly, to plating systems and instruments for stabilizing and/or fusing boney structures, such as the spine.

### BACKGROUND INFORMATION

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (lateral bending to either left or right side) and rotation (twisting of the shoulders relative to the pelvis).

The spine of most human adults consists of 24 connected bones called vertebrae. The cervical vertebrae begin at the base of the skull. Seven vertebrae make up the cervical spine, which are abbreviated C1, C2, C3, C4, C5, C6 and C7. The cervical vertebrae are smaller in size compared to other spinal vertebrae. The purpose of the cervical spine is to contain and protect the spinal cord, support the skull, and enable a wide range of head movement. The vertebrae allow the head to rotate side to side, bend forward and backward.

The intervertebral spacing (between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae, during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal cord to extend out of the spine, between neighboring vertebrae, without being squeezed or impinged by the vertebrae.

Frequently cervical spine disorders require surgery to relieve painful symptoms. One of the contributing factors associated with most spine disorders is the dehydration of the intervertebral disks, which act as a cushion between adjacent vertebrae. Over time these disks can dry out and become flattened, causing the vertebrae to lose height and its healthy resilience. The degeneration of the disks allow the vertebrae get closer together and cause nerve irritation, which usually stems from a ruptured disc, bone spurs or stenosis. Vertebral motion (neck movement) results in chronic pain.

Cervical fusion has become an accepted procedure to relieve the pressure on one or more nerve roots, or on the spinal cord. It involves the stabilization of two or more vertebrae by locking (fusing) them together in a desired spacing and orientation. The fusion restores the proper distance between the vertebrae which aids in preventing nerve irritation.

The cervical spine may be approached by the surgeon anteriorly, which refers to the front of the patient. The surgeon reaches the cervical spine through a small incision in the front of the neck. After retracting neck muscles, the surgeon often removes the affected intervertebral disk, which takes the pressure off the nerves or spinal cord. This is procedure is known as decompression. The surgeon then may replace the removed disk with a bone graft or interbody fusion device (such as a cage) to aid in the fusion of adjacent vertebrae and in the restoration of the distance between the vertebrae. The surgeon then may use various types of plates which provide extra force on the graft (or interbody fusion device) and support the neck to ensure that the bones fuse adequately. Frequently, holes may be drilled or tapped in the bone to allow for attachment of a plate using a bone screw or other fastener. The plate is placed against two or more adjacent vertebrae and bone fasteners are used to secure the plate in place.

One of the problems associated with the fusion of cervical vertebrae is the tendency of the screws or other fasteners to loosen over time. As the fasteners or screws loosen the plate is not able to support or maintain the proper orientation of the vertebrae. The plate and other associated implants, which are no longer secure, can cause irritation and even trauma to local tissue structures. Another problem associated with the fusion of cervical vertebrae is the tendency of the bones or vertebrae not to fuse together, which may require an additional surgical procedure to correct. Poor fusion may also result from subsidence of a bone graft or an interbody device placed between vertebrae (or other boney structure). Subsidence occurs when the bone graft or interbody device that is placed between to vertebral end plates sinks or settles into the vertebral end plates. When subsidence occurs, the extra force or pressure placed on the interbody device or bone graft by a plate may be reduced to nothing. If little load is transferred to the bone (or bone graft), the bone may become weaker, resulting in a poor fusion. Inadequate fusion or healing of bones is prevalent in other areas of the body besides the cervical spine, such as, the lumbar and thoracic spine, long bones as well as other boney structures.

### BRIEF DESCRIPTION OF THE DRAWINGS

FIG. 1. is a perspective view illustrating one embodiment of the plate of the present disclosure;

FIG. 2 is a cross sectional side view of the plate of FIG. 1;

FIG. 3 is a front view of the plate of FIG. 1;

FIG. 4A is a front view illustrating a possible first position of the plate of FIG. 1;

FIG. 4B is a cross sectional side view illustrating a possible first position of the plate of FIG. 1;

FIG. 5A is a front view illustrating a possible second position of the plate of FIG. 1;

FIG. 5B is a cross sectional side view illustrating a possible second position of the plate of FIG. 1;

FIG. 6 is side view illustrating one possible embodiment of a plate holder instrument and a cross sectional side view of the plate of FIG. 1;

FIG. 7 is an oblique view illustrating one possible embodiment of a gauge that may be incorporated in the plate of FIG. 1;

FIG. 8 is an anterior view illustrating one possible embodiment of a cover that may be incorporated in a plate of FIG. 1;

FIG. 9 is an anterior view of the cervical spine illustrating the plate of FIG. 1 implanted to a first and second vertebrae;

FIG. 10A is a front view of another possible embodiment of a plate illustrating one possible embodiment of indicator features that may be incorporated in the plate;

FIG. 10B is a cross sectional side view of the plate of FIG. 10A;

FIG. 11 is a front view of a plate with another possible embodiment of indicator features that may be incorporated in the plate;

FIG. 12A is a front view illustrating one possible embodiment of a multi-level plate;

FIG. 12B is a cross sectional side view of the multi-level plate shown in FIG. 12A;

FIGS. 13A-13F are detailed oblique views of possible embodiments of slider segments that may be incorporated into a plate;

FIG. 14A is a front view of a plate which may incorporate various slider members;

FIGS. 14B-14F are cross sectional views taken along line B-B of FIG. 14A illustrating alternative embodiments of a sliding and receiving segments that may be incorporated in the plate of FIG. 1;

FIG. 15 is a cross sectional side view of another possible embodiment of a plate incorporating one possible embodiment of a slider segment;

FIG. 16 is a cross sectional side view of another possible embodiment of a plate incorporating another possible embodiment of a slider segment; and

FIG. 17 is a front view of another possible embodiment of a plate incorporating another possible embodiment of a slider segment.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

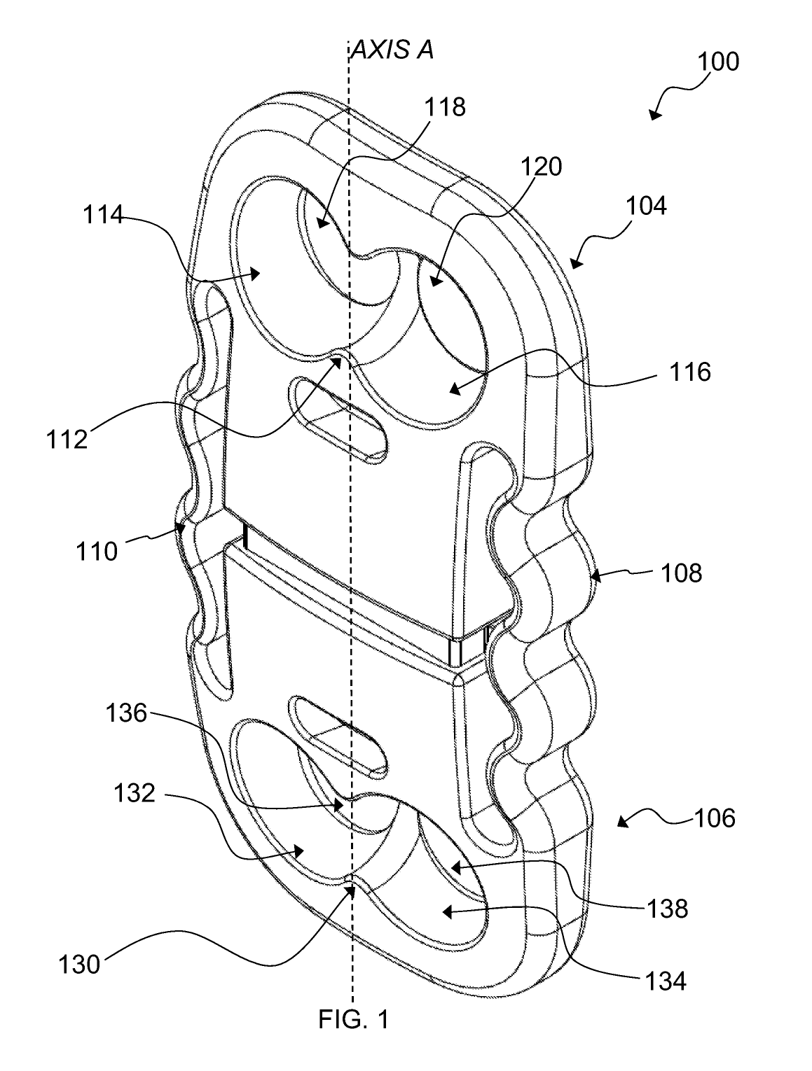

Turning now to FIG. 1, there is presented a perspective view of one possible embodiment of a plate 100. The plate 100 may extend generally along a longitudinal Axis A and may include a first plate member 104, a second plate member 106 and one or more compression biasing members 108 and 110. As will be discussed in greater detail below, the plate 100 may be fastened to one or more adjacent (or non adjacent) boney structures, such as vertebrae, and the compression biasing members 108 and 110 may force the vertebrae closer together. In some embodiments, the compression biasing members 108 and 110 may aid in compressing a graft or an implant located between the adjacent vertebrae to promote fusion. The plate 100 may also be used to stabilize boney fractures which may or may not have an implant or graft in-between to promote fusion or improve healing. For example, the compression biasing members 108 and 110 may force long bone structures on opposing sides of a fracture together to promote fusion. Accordingly, the first plate member 104, the second plate member 106 and the compression biasing members 108 and 110 may interact to provide the proper stabilization forces required to support or fuse boney structures.

In certain embodiments, a proximal end portion of the compression biasing member 108 may be secured to a first side wall of the first plate member 104. A distal end portion of the compression biasing member 110 may be secured to a first side wall of the second plate member 106. In certain embodiments the proximal end portion of the compression biasing member 110 may be secured to a second side wall of the first plate member 104 and a distal end portion of the compression biasing member 110 may be secured to a second side wall of the second plate member 106. Many different mechanical, thermal or chemical attachment means that are well known in the art may be used to secure the first and second compression biasing members 108 and 110 with the first and second plate members 104 and 106. For example, methods such as welding, press fitting, pinning, screws, adhesives and insert molding may be used. In other embodiments compression biasing members 108 and 110 may be a unitary and integral part of the first plate member 104 and second plate member 106. A unitary plate or single piece plate may have several advantages over a multiple component plate design that has several components that must be assembled before implantation. For example, a unitary plate design may be more structurally rigid, have more uniform properties and provide for increased compression than a multiple component type plate design.

The first plate member 104 may include a first recess 112 having a first portion 114 and a second portion 116 and a first bore 118 aligned with the first portion 114 and a second bore 120 aligned with the second portion 116. The first recess 112 may extend partially into a top surface of the first plate member 104 and the first and second bores 118 and 120 may extend through the first plate member 104. The first recess 112 may be dimensioned to receive two bone screws each having a head portion and a shaft portion (not shown). The first portion 114 may receive the head of the first bone screw and the second portion 116 may receive the head of the second bone screw (not shown). The first and second bores 118 and 120 may receive the shaft portion of the first and second bone screws, respectively.

The second plate member 106 may include a second recess 130 having a third portion 132, a fourth portion 134, a third bore 136 that aligned with the third portion 132 and a fourth bore 138 aligned with the fourth portion 134. In certain embodiments a side wall of the first and second recesses 112 and 130 may have a concave surface that receives a head of a bone engagement member (not shown). The second recess 130 may extend partially into a top surface of the second plate member and the third and fourth bores 136 and 138 may extend through the second plate member 106. The recess 130 may be dimensioned to receive a third and a fourth bone screws each having a head portion and a shaft portion (not shown). The third portion 132 may receive the head of the third bone screw and the fourth portion 134 may receive the head of the fourth bone screw. The third and fourth bores 136 and 138 may receive the shaft portion of the first and second bone screws, respectively. The bone screws and the attachment of the plate 100 to a pair of vertebrae will be explained in greater detail later.

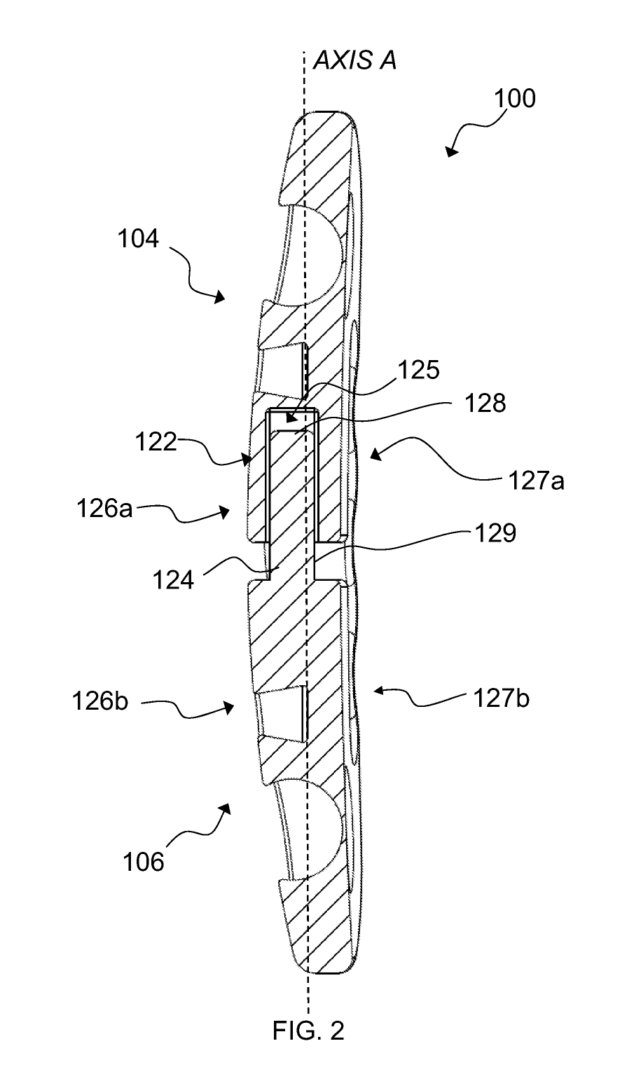

Referring to FIG. 2, a cross sectional side view of the plate 100 is shown. In certain embodiments, the first and second plate members 104 and 106 may have a convex anterior surface 126a and 126b (respectively) and a generally concave posterior surface 127a and 127b (respectively) that extend along the longitudinal Axis A. In certain embodiments the first plate member 104 may be slidably mated with the second plate member 106. The first plate member 104 may have a receiver segment 122 that defines a longitudinal portal 125. The longitudinal portal 125 may be open on a first end and closed on an opposite second end. The longitudinal portal 125 may be dimensioned to at least partially receive a slider segment 124 of the second plate member 106. In certain embodiments the slider segment 124 and the longitudinal portal 125 may be dimensioned to allow for a gap which may allow the first plate member 104 to pivot relative to the second plate member 106. In certain embodiments, the slider segment 124 may be generally rectangular in shape and may have a proximal and distal end portions 128 and 129. The proximal end portion 128 may have a reduced profile or lead in to aid insertion into the longitudinal portal. The slider segment 124 may slide within the longitudinal portal 125 as the plate 100 is expanded or compressed. Other configurations for the slider segment 124 and the longitudinal portal 125 are possible, as will described in greater detail later.

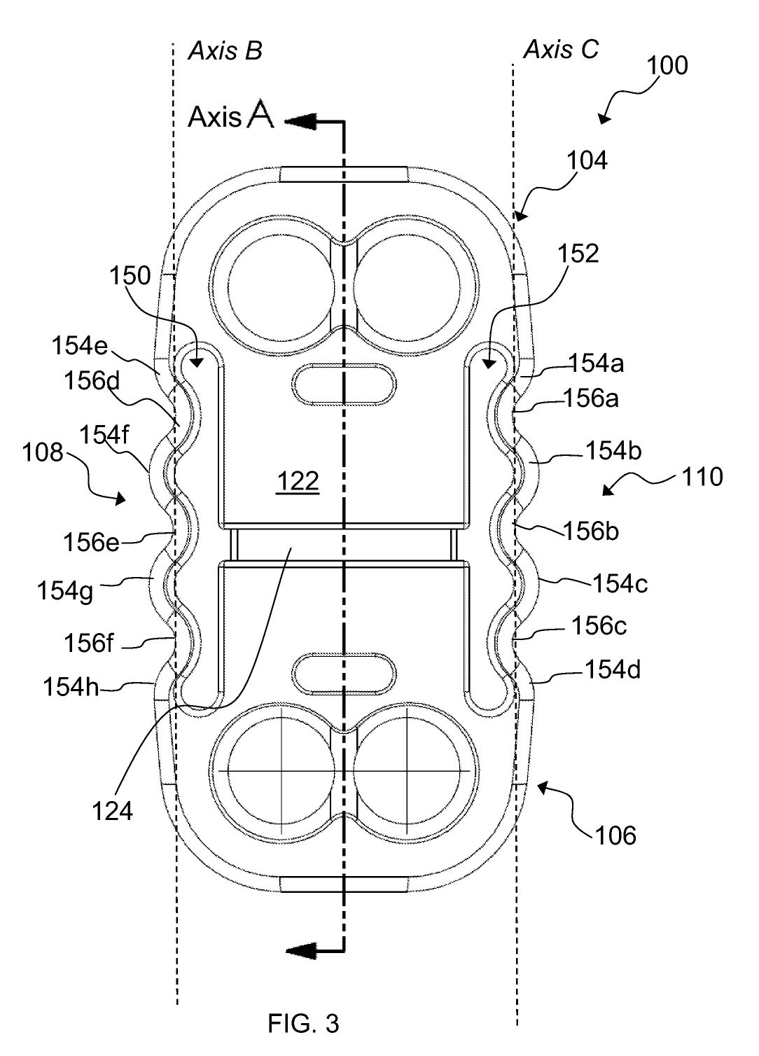

Referring now to FIG. 3, as illustrated the first and second compression biasing members 108 and 110 may extend longitudinally along Axis B and Axis C (respectively) which may run generally parallel to the longitudinal axis of the plate (Axis A). The first and second compression biasing members 108 and 110 may be laterally spaced apart from the receiving segment 122 and the slider segment 124. In certain embodiments, the first compression biasing member 108 and the first and second plate members 104 and 106 may form a continuous wall that defines a first window 150. In certain embodiments, the second compression biasing member 110 and the first and second plate members 104 and 106 may form a continuous wall that defines a second window 152. The first and second windows 150 and 152 may allow a surgeon an improved view a boney structure to which the plate 100 is attached. The first and second windows 150 and 152 may enable a surgeon to have a better view of a graft or implant (not shown) which may be positioned between a first and second vertebrae (not shown) and determine if the plate, graft and/or implant has been placed correctly. Post operatively a surgeon may take an X-ray of the plate 100 and a boney structure, such as vertebrae, to determine the progress of fusion or healing. The first and second windows 150 and 152 may enable a surgeon to have a better view to determine if the bone is healing or fusing properly.

In certain embodiments the first and second compression biasing members 108 and 110 may have a circular or partially circular cross section (such as an oval, race track or semi-circle) and may have a wall thickness that is generally equivalent to an overall wall thickness of the plate 100. For example the thickness of the first and second compression biasing members 108 and 110 may be less than the wall thickness of the plate 100, so that a top surface of first and second compression biasing members 108 and 110 does not extend above a top surface of the plate 100. A smaller wall section or thickness of the first and second compression biasing members 108 and 110 may minimize an overall profile of the plate which may reduce interference with neighboring anatomy. For example, in a cervical plate application, a thicker plate may interfere with the esophagus of a patient.

In certain embodiments the first and second compression biasing members 108 and 110 may include a plurality of successive waves in which the waves include alternating wave crest 154a-154h and wave trough portions 156a-156f. The wave crest 154a-154h and wave trough portions 156a-156f may include curved segments that travel generally within a first plane of the plate 100. The wave crest 154a-154h and wave trough portions 156a-156f may all travel generally within the same plane as one another, unlike a helical-type or coil spring. The wave crest 154a-154h and wave trough portions 156a-156f may include full or partial curved segments.

The compression biasing members 108 and 110 may offer several advantages over conventional coil springs. The compression biasing members 108 and 110 do not extend in a helical fashion around a central axis, but extend in a generally longitudinal direction. A core or coil is created when a spring extends in a helical fashion around a central axis. This core or coil takes up valuable space, especially for a small implant such as an orthopedic plate. Also the larger the force required, the larger the coil and more space that is consumed. The compression biasing members 108 and 110 are designed to exert a maximum force while consuming a minimum amount of space, not only to minimize the size and thickness of the plate, but to allow a surgeon a better view the anatomy to which the plate is attached. Another advantage of compression biasing members 108 and 110 is that their low profile does not interfere with neighboring anatomy which may be impinged. Coils tend to collapse on themselves, thus trapping or impinging neighboring anatomy which may cause pain or damage to a patient. Tissue may also have the tendency to grow within the core or in-between the coils, thus interfering with the function of the plate. The wave crest 154a-154h and wave trough portions 156a-156f do not have a core and are designed not to compress against each other which may impinge or trap tissue that is near the plate 100. A coil type design may detach from the plate, which may prevent the plate from functioning properly and may injure the patient.

In certain embodiments the first and second compression biasing members 108 and 110 may be manufactured from memory shape materials such as nitinol, elastomers or polymers. In other embodiments the first and second compression biasing members 108 and 110 may be manufactured from titanium, stainless steel or other biocompatible materials. The first and second compression biasing members 108 and 110 may be cast, machined, molded or manufactured from any combination of commonly known manufacturing processes. In certain embodiments the first and second plate members 104 and 106 may be manufactured from the same or dissimilar materials. The first and second plate members 104 and 106 may be manufactured from as nitinol, titanium, stainless steel, elastomers, polymers or other biocompatible materials. The first and second plate members 104 and 106 may be cast, machined, molded or manufactured from any combination of commonly known manufacturing processes.

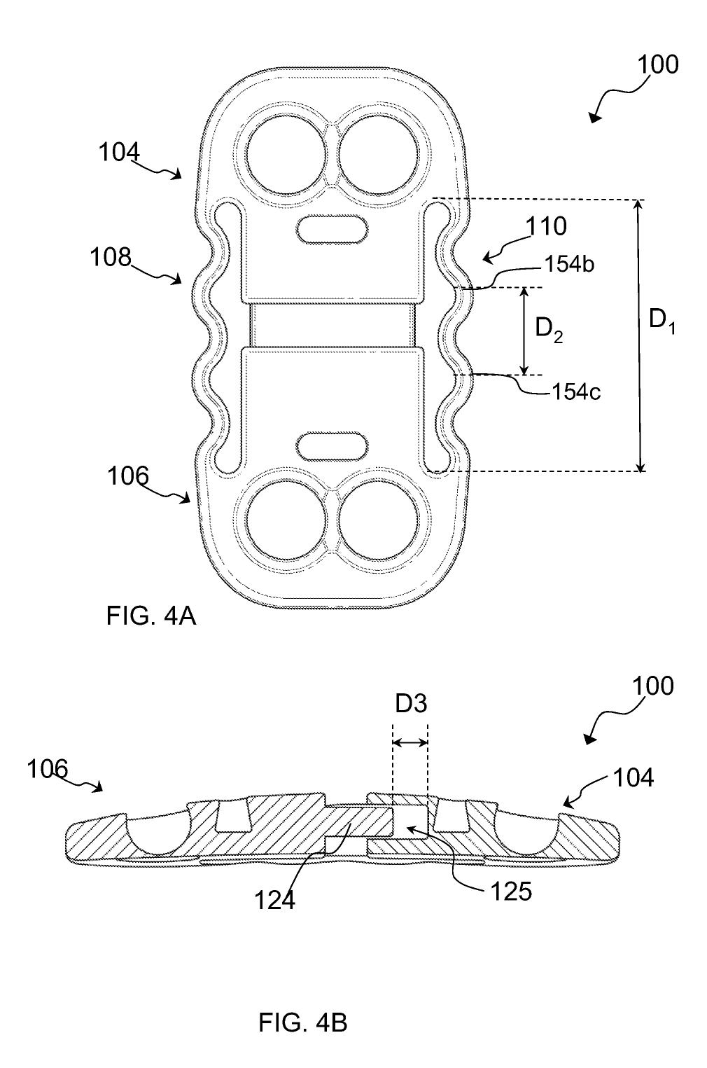

Turning now to FIGS. 4A and 4B a front view and a cross sectional side view of the plate 100 is shown. FIG. 4A is a front view of plate 100 illustrating the first plate member 104 and the second plate member 106 in an expanded first position. A force may be applied to the plate 100 to expand the plate 100 a certain distance or to achieve a certain force prior to implanting or attaching the plate 100 to a pair of vertebrae. When a distraction force is placed on the plate, the force may cause the compression biasing members 108 and 110 to elongate. The distance D1 may be used to represent the overall length of the compression biasing members 108 and 110 when the plate 100 is expanded. The distance D2 between adjacent crest portions 154b and 154c (and/or trough portions), may also increase as the plate 100 is distracted to the expanded first position.

FIG. 4B is a cross sectional side view of the plate 100 illustrating the expanded first position. The position of the slider segment 124 relative to the longitudinal portal 125 may change depending on the distance the plate 100 is expanded. The distance D3 may represent the distance between the first plate member 104 and the second plate member 106 when the plate 100 is in the expanded first position. The distance D3 may also represent the amount of travel before the first plate member 104 and the second plate member 106 abut each other and prevent the plate 100 from contracting any further. One skilled in the art would appreciate that D1, D2 and D3 may vary greatly depending on the distance between vertebrae, the force required to compress the vertebrae, the size of the bone graft or interbody device used and level of possible subsidence, etc.

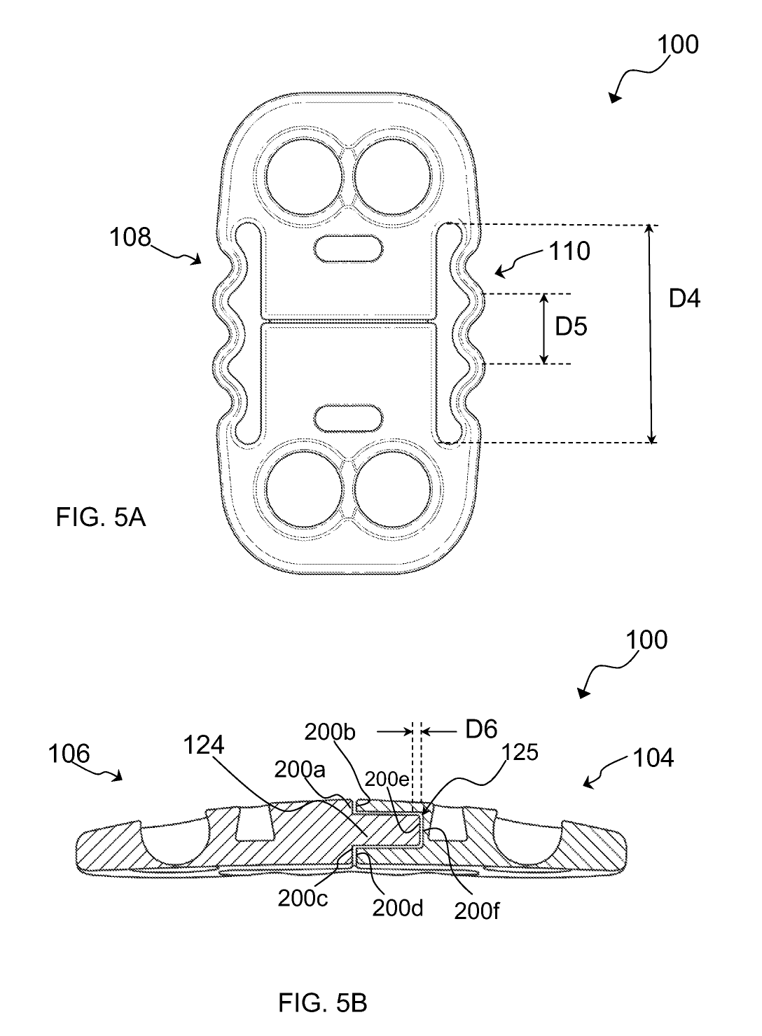

Referring now to FIGS. 5A and 5B a front view and a cross sectional side view of the plate 100 is shown in a possible second position. After the plate 100 is inserted and fixed to a pair of vertebrae (or other boney structure) the distraction force may be removed. Once the distraction force is removed, the plate 100 may transfer a compressive force to the vertebrae as the first and second compression biasing members urge the first and second plate members 104 and 106 closer together. The plate 100 may actively compress the vertebrae together, which may result in an improved fusion. The plate 100 may also compress a graft or an implant (not shown) that is located between the two vertebrae, which may help prevent expulsion of the graft or implant. The compression biasing members 108 and 110 may be capable of exerting a compression force on the vertebrae (or graft/implant) which is greater than a force normally acting on the vertebrae when a patient is in a standing position. In certain embodiments the compression biasing members 108 and 110 may exert a compressive force of less than 10 lbs to more than 50 lbs.

The distances D1 and D2 may decrease causing the first and second plate members 104 and 106 to move closer together resulting in a fourth and fifth positions, as represent by D4 and D5. The distance between the first plate member 104 and the second plate member 106, as represented by D6, may also decrease when the distraction force is removed from the plate 100. In certain embodiments the distances D4, D5 and D6 may be less than D1, D2 and D3 respectively, but may still be greater than these corresponding distances when the plate 100 is in a pre-expanded or neutral position.

After the plate 100 is attached to the adjacent vertebrae, settling may occur, as the vertebrae move closer together. This may cause the first and second plate members to move closer together. The plate 100 may have one or more abutment surfaces 200a-200f which may prevent the first and second the first plate members 104 and 106 from moving closer together. The abutment surfaces may prevent the vertebrae from collapsing together and may help maintain disc height (the distance between adjacent vertebrae). In some embodiments an end wall 200e of the slider segment 124 may contact a back wall 200f of the longitudinal portal 125 to prevent the first and second plate members 104 and 106 from moving closer together. In other embodiments a bottom surface 200b and 200d of the first plate member 104 may contact a top surface 200a and 200c of the second plate member 106 to prevent further compression of the plate. The geometry and properties of the compression biasing members 108 and 110 may also resist compression forces and prevent the plate 100 from compressing beyond a certain point.

The compression biasing members 108 and 110 may be placed in tension so the plate 100 compresses one or more boney structures to which the plate 100 is attached. As the distance the compression biasing members 108 and 110 are expanded or stretched increases, the resulting compressive force created may also increase. The compression biasing members 108 and 110 may follow the equation “F=−k×”, wherein “F” represents the force on the compression biasing members 108 and 110, “k” is a spring constant of compression biasing members 108 and 110 and “x” is the displacement of compression biasing members 108 and 110.

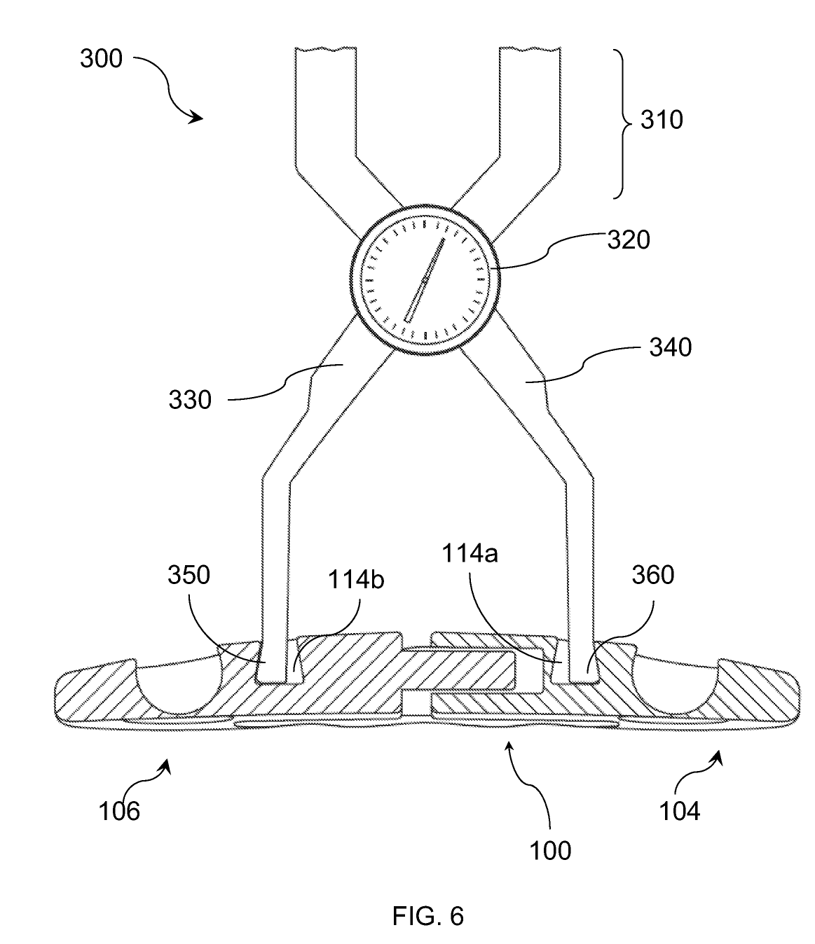

Now turning to FIG. 6 a cross section of the plate 100 is shown with one possible embodiment of a plate holding instrument 300. The first and second plate 104 and 106 members may have one or more instrumentation slots 114a and 114b dimensioned to receive the plate holding instrument 300 which may spread the first and second plate members 104 and 106 apart. The plate holding instrument 300 may hold the plate 100 in tension until the plate 100 is secured to a boney structure. In certain embodiments instrumentation slots 114a and 114b may extend partially into a front surface of the first and second plate members 104 and 106, respectively. In other embodiments instrumentation slots 114a and 114b may extend completely through the front surface of the first and second plate members 104 and 106. The instrumentation slots 114a and 114b may have a racetrack or oval shape, although other configurations are also possible, such as circles, rectangles squares, dovetails, keyholes, etc. Although the instrumentations slots 114a and 114b are shown as being located on the top surface of the plate 100, the instrumentations slots 114a and 114b may be located on a first and second side surfaces of the first and second plate members 104 and 106.

In certain embodiments plate holding instrument 300 may have an actuator mechanism 310, a force/distance indicator 320 and a pair of plate attachment arms 330 and 340. The distal end of attachment arms 330 and 340 may have two or more feet 350 and 360 which may be dimensioned to releasably fit within instrumentation slots 114a and 114b. The actuator mechanism 310 may include a squeeze handle in which the attachment arms 330 and 340 rotate about a pivot point, however other mechanisms are also possible, such as rack and pinion or worm gear mechanisms. When the handle is squeezed, attachment arms 330 and 340 may pivot and move away from each other forcing the first plate member 104 and the second plate member 106 away from each other to expand the plate 100. The actuator mechanism 310 may include a locking mechanism (not shown), such as a ratchet mechanism on the attachment arms 330 and 340, which maintains the tension on the plate without having to continuously apply force to the arms 330 and 340. As the plate is expanded by instrument 300, force/distance indicator 320 may measure and indicate the force required to expand the plate or a distance the plate 100 has expanded. In certain embodiments the force/distance indicator 320 may measure both the force and the distance the plate has expanded. In other embodiments force/distance indicator 320 may also measure and indicate the force and/or distance required to compress the plate 100.

Referring to FIG. 7 another embodiment of a plate 400 is shown which may indicate a force exerted on the plate 400. Plate 400 may have one or more strain gauges 402 attached to one or more compression biasing members 408 and 410. After the plate 400 is attached to the vertebrae the strain gauge 402 may indicate a strain or a force acting on the compression biasing members 408 and 410. The surgeon may want a specific force on the plate 400 which may be transferred to a boney structure. In this situation the surgeon would compress or extend plate 400 to the required reading on the strain gauge 402 and then attach plate 400 to the boney structure (not shown). The strain gauge 402 may be bonded to the plate using adhesives, solvent bonding, welding or soldering.

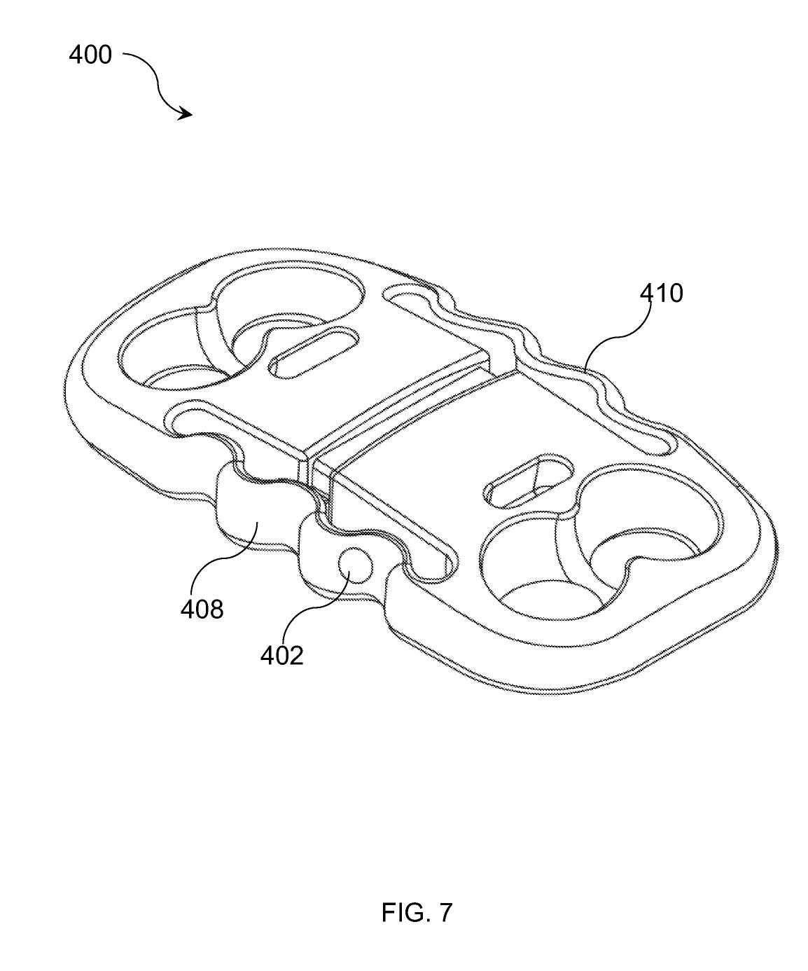

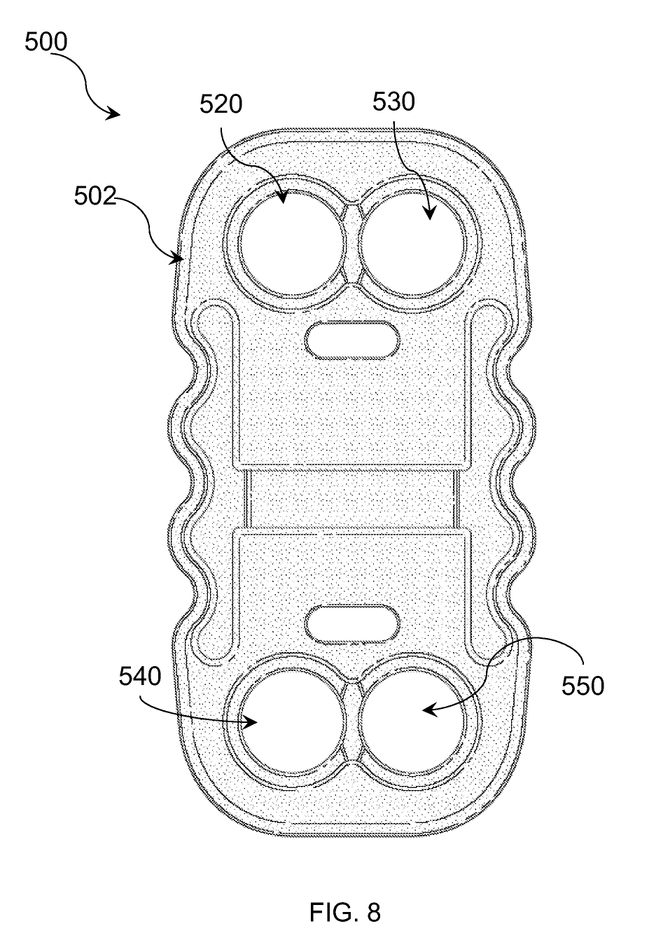

Referring now to FIG. 8, in certain embodiments it may be desirable for the plate 500 to have an outer covering 502. The covering 502 may be incorporated in any of the embodiments described herein. The covering 502 may wrap around the entire plate 500 (as shown) or may cover only certain areas of the plate 500. The covering 502 may allow for open areas 520, 530, 540 and 550 for the placement of bone anchors, such as screws. In certain embodiments the cover 500 may include a weave or braid construct manufactured from fabric type materials such as nylon or polyester or the covering 502. In other embodiments the cover may be an injection molded elastomeric material, such as silicone or neoprene. Several assembly methods may be used to attach cover 500 to plate 100 such as hot dipping, a stretch fit, over molding, or snap fits. Additional components may also be used to assemble the covering 502 to plate 500 such as screws, tacks, fibers or adhesives. In certain embodiments, the covering 502 may provide for a dampening effect, act to resist compressive or extension forces or prevent tissue in-growth around moving components of the plate 100. In certain embodiments the covering 502 may prevent any moving components of the plate 500 from impinging on neighboring anatomy. In some embodiments, the covering 502 may be composed of a radiolucent material and may have radio opaque markers (not shown).

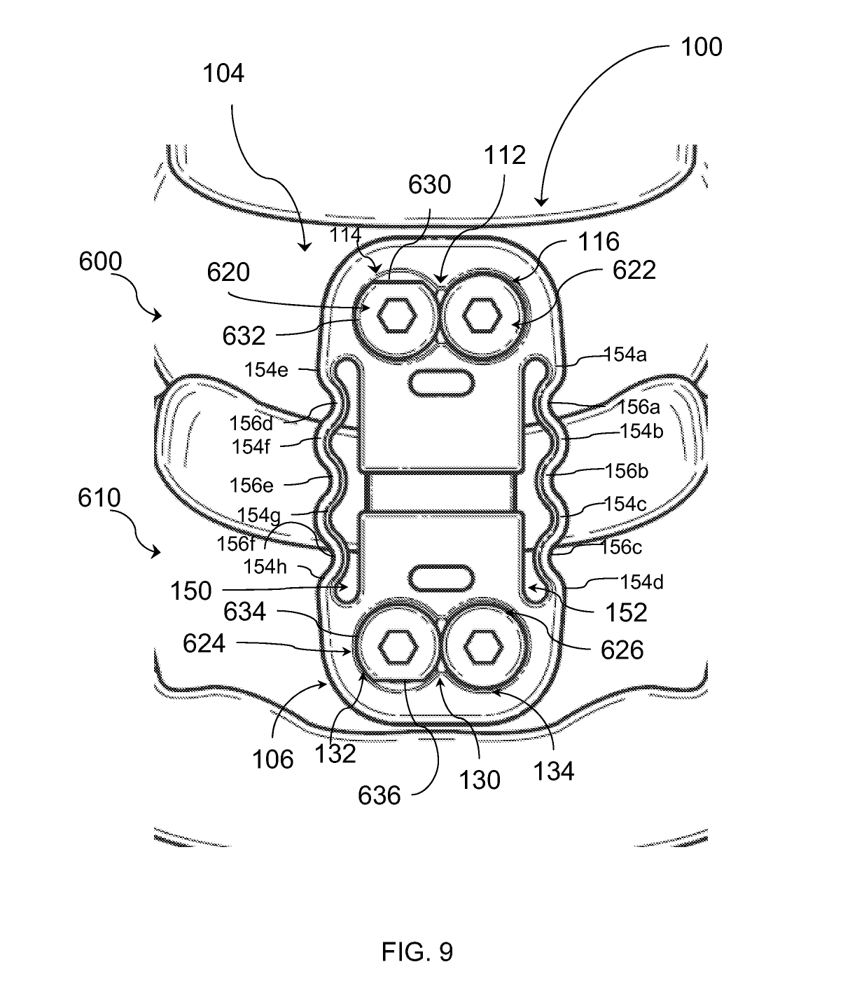

Referring to FIG. 9, the plate 100 is shown implanted to a pair of cervical vertebrae 600 and 610, however it is understood that the plate 100 may be implanted to non adjacent vertebrae or to other bony structures. A proper size plate 100 for the patient's anatomy may be determined and placed over the two adjacent vertebrae 600 and 610, such that the first recess 112 is located generally in the middle of the first vertebrae 600 and the second recess 130 is located generally in the middle of the second vertebrae 610. The plate 100 may be expanded using an instrument, such as the plate holder 300 described above (not shown). As the plate 100 is expanded, the axial distance between adjacent wave crest 154a-154h and wave trough portions 156a-156f may be increased. Once the desired amount of force or tension on the plate 100 is achieved, as measured by the plate holder 300, strain gauge 402 or other means (as described above and shown in FIGS. 6 and 7), the first plate member 104 may be secured to the first vertebra 600 with a first and second bone anchors 620 and 622 and the second plate member 106 may be secured to the second vertebra 610 with a third and fourth bone anchors 624 and 626.

In certain embodiments, the plate 100 may have a locking mechanism to prevent the bone anchors from backing out of the vertebrae 600 and 610 or the plate 100. For example, in some embodiments, the first and second bone anchors 620 and 622, may each have a head portion which may be inserted such that the respective heads compress and lock against one another and the plate 100. A third and fourth bone anchors 624 and 626 may also be inserted such that the respective heads compress and lock against one another.

In certain embodiments a first and second portions 114 and 116 of the first recess 112 may receive the first and second bone anchors 620 and 622, respectively. The first portion 114 may receive a head of the first bone anchor 620 and the second portion 116 may receive a head of the second bone anchor 622. The first and second bores (as shown in FIG. 1) may receive a shaft portion (not shown) of the first and second bone anchors, respectively. The first bone anchor 620 may have a non cam surface 630 and a cam surface 632. The first bone anchor 620 may be secured to the first vertebra and adjusted so that the non-camming surface 630 generally faces the second portion 116. The adjusting of the non camming surface 630 may allow the second bone anchor 622 to be fully inserted into the first vertebra 600 without interfering with the first bone anchor 620. The first bone anchor 620 may be adjusted or rotated so that the cam surface 632 engages the surface of the head of the second bone anchor 622, locking the adjacent bone anchors 620 and 622 against one another and the plate 100.

In certain embodiments the third and fourth bone anchors 624 and 626 may be locked in a similar fashion. The third and fourth portions 132 and 134 of the second recess 130 may receive the third and fourth bone anchors 624 and 626, respectively. The third portion 132 may receive a head of the third bone anchor 624 and the fourth portion 134 may receive a head of the fourth bone anchor 626. The third and fourth bores (as shown in FIG. 1) may receive a shaft portion (not shown) of the third and fourth bone anchors 624 and 626, respectively. The third bone anchor 624 may have a cam surface 634 and a non cam surface 636. The third bone anchor 624 may be secured to the second vertebra 610 and adjusted so that the non-camming surface 636 generally faces the fourth portion 134 of the second recess 130. This may allow the fourth bone anchor 626 to be fully inserted into the second vertebra 610 without interfering with the third bone anchor 624. The third bone anchor 624 may be adjusted so that the cam surface 634 engages the surface of the head of the fourth bone anchor 626, locking the adjacent bone anchors 624 and 626 against one another and the plate 100. Other locking mechanisms may be used to lock the bone anchors 620, 622, 624 and 626 relative to each other and to the plate 100. These locking mechanisms may include additional camming screws, tabs or clip members.

Once the plate 100 is secured to the vertebrae 600 and 610, the plate holding instrument, such as the instrument 300, may be removed. Once the plate holding instrument is removed the compression biasing members 108 and 110 may compress moving the first and second plate members 104 and 106 and the first and second vertebrae closer together. In certain embodiments the plate 100 may compress an implant of bone graft (not shown) which may be placed in-between the first and second vertebrae 600 and 610. As previously discussed, the first and second windows 150 and 152 may enable a surgeon to have a better view of the graft and determine if the plate, graft and/or implant has been placed correctly or the progress of healing post operatively.

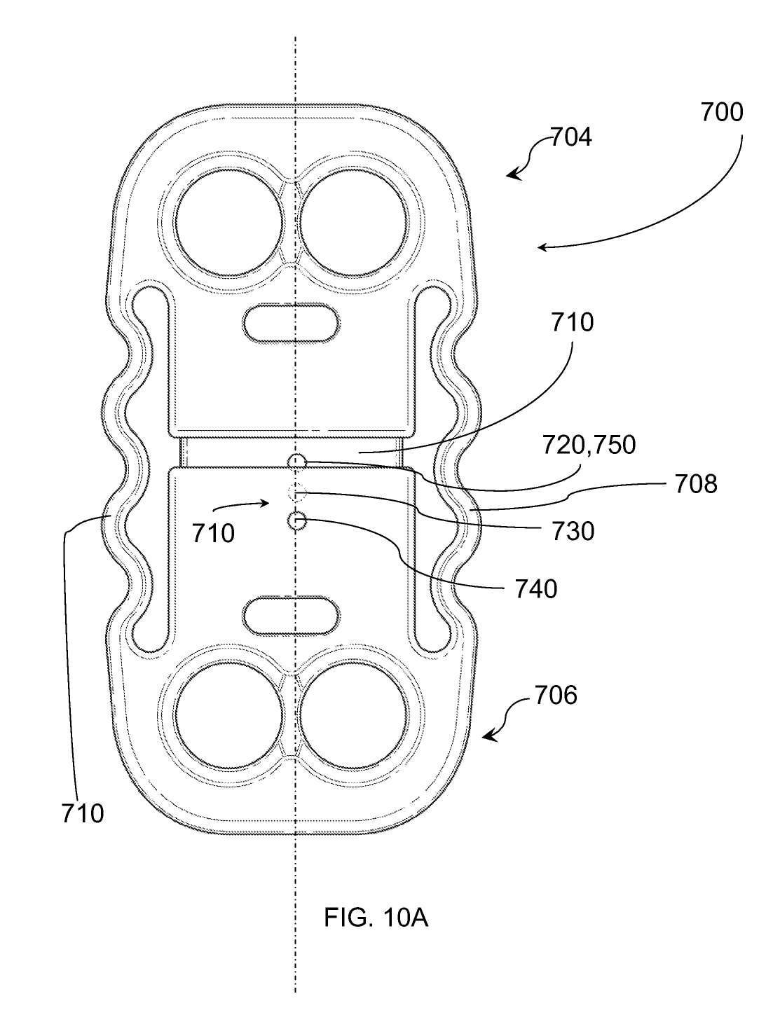

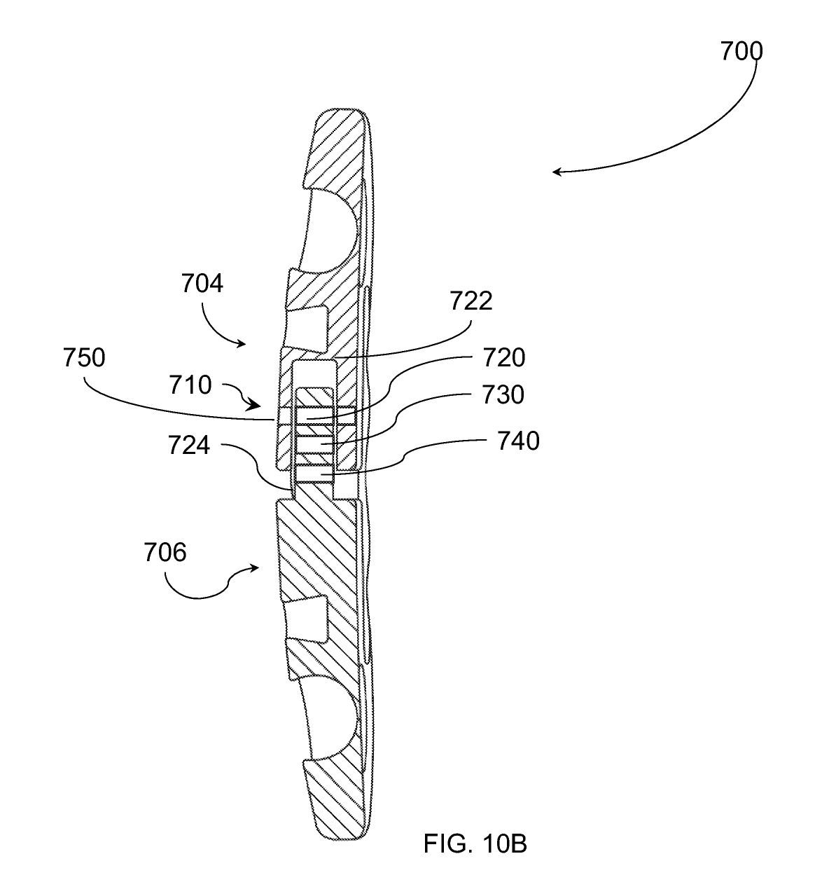

After the plate 100 is implanted into a patient it may be desirable for the surgeon to determine how much the plate has settled or moved over time without operating on the patient. FIGS. 10A and 10B illustrate one possible embodiment of a plate 700 with alignment features 710 in which a non invasive method such as X-ray or fluoroscopy may be used to determine movement of the plate within the patient. In certain embodiments plate 700 may be similar to plate 100, however plate 700 may include an inner surface that define a series of alignment holes 720, 730 and 740 which may extend through an anterior (front) surface of the second plate member 706. Holes 720, 730 and 740 may be positioned along a common longitudinal axis and may be located generally in the center of the second plate member 706. A first plate member 704 may have one or more alignment holes 750 that may extend through an anterior (front) and a posterior (back) surface of the first plate member 704 of the plate 700.

In certain embodiments the plate may be composed of a radio opaque material which is visible on an X-Ray or fluoroscopy image. FIG. 10A illustrates what a surgeon may see on an X-ray or fluoroscopy image. The hole 750 of the first plate member 704 may be aligned with hole 720 of the second plate member. The holes 730 and 740 may not be differentiated from the plate 100, but the overlapping holes 720 and 750 would be visible due to the contrast with the radio opaque material of the plate 700. The surgeon may then compare the hole alignment on the post surgery X-ray or fluoroscopy image with the image that was taken just after implantation. For example, if the image taken immediately after implantation displayed the hole 750 not aligned or overlapping with the hole 720 and post operatively the image showed the hole 720 aligned with the hole 740 the surgeon could determine that the plate compressed a certain distance. The distance between holes 720, 730 and 740 may correspond to an approximate distance or to an approximate force required to move the first plate member 704 a certain distance relative to the second plate member 706.

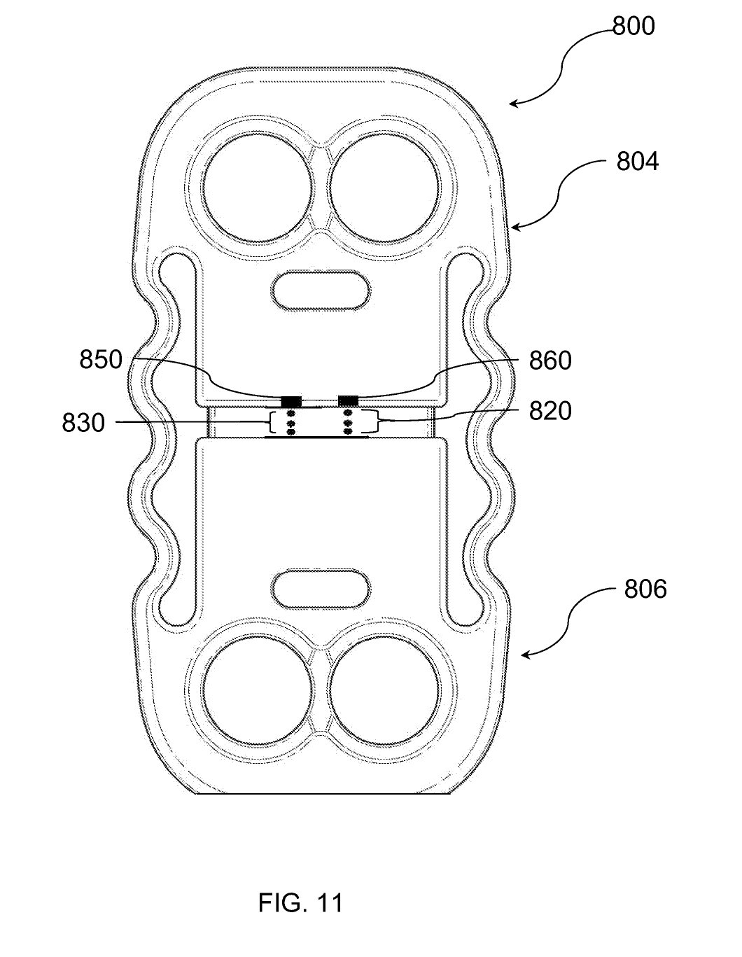

In other embodiments the second plate member 706 may have more or less than three holes depending on the size of the plate 700 and the X-ray or fluoroscopy resolution required in determining changes in forces or distances. In certain embodiments first plate member 704 may have more than one hole depending on the size of the implant and the resolution required in determining changes in forces or distances. For example, FIG. 11 shows an alternative embodiment of a plate 800 which has a pair of alignment markers 850 and 860 in a first plate member 804 and a first and second series of alignment markers 820 and 830 in a second plate member 806. As the plate 800 compresses the alignment markers 850 and 860 may move in relation to the series of alignment markers 820 and 830. The alignment markers 850 and 860 may have different shapes, sizes or geometries in relation to the series of alignment markers 830 and 820 in order to be discernable on an X-ray or fluoroscopy image.

In certain embodiments the plate 700 or 800 may be composed of radio opaque material, such as titanium and the alignment holes or markers may be filled with a radiolucent insert, such as a polymer or bone filler, that does not show up or is differentiable from the plate 700 or 800 on X-ray or fluoroscopy images. In other embodiments, the plate 700 or 800 may be composed of a radio lucent material, such as PEEK polymer and the alignment holes or markers may be filled with a radio opaque insert such as titanium. An insert may also allow the alignment hole size to be increased without sacrificing strength. A radiolucent bone filler insert may allow for bone in growth into the plate to aid in vertebral fusion. In certain embodiments the alignment holes may be larger and/or have different geometries (such as a rectangle, oval, racetrack, or cross) to allow the surgeon to easily distinguish between different holes.

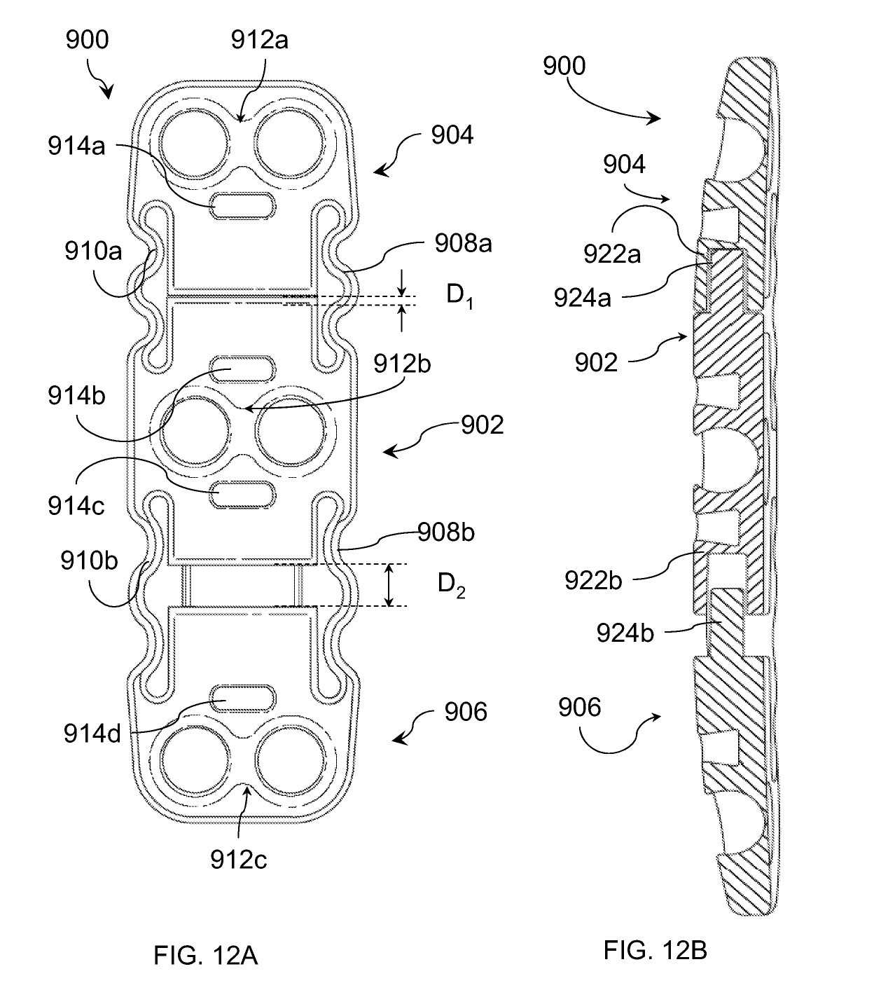

Turning now to FIGS. 12A and 12B, there is presented an embodiment of a multi-level plate 900 that may be used to stabilize or fuse three or more vertebrae. The multi-level plate 900 may be similar to the plate 100, except multi level plate may include a third plate member 902 located between a first plate member 904 and a second plate member 906. The first plate member may include an instrumentation slot 914a and a first recess 912a for two or more bone anchors (not shown), similar to the plate 100. The first plate member 904 may be coupled to the third plate member 902 by compression biasing members 908a and 910a. The third plate member 902 may include instrumentation slots 914b and 914c and a second recess 912b for two or more bone anchors (not shown). The third plate member 902 may be coupled to the second plate member 906 by compression biasing members 908b and 910b. The second plate member 906 may include an instrumentation slot 914c and a third recess 912c for two or more bone anchors (not shown), similar to the plate 100. The compression biasing members 908a, 908b, 910a and 910b may be the same or similar in structure and function as the compression biasing members 108 and 110 of the plate 100, as previously described.

Referring to FIG. 12B, in certain embodiments, the third plate member 906 may include a first slider member 924a which may be dimensioned to slide within a receiver member 922a of the first plate member 904. The first slider member 924a may have a generally rectangular shape, similar to the plate 100 as described previously. The second plate member 906 may include a second slider member 924b which may have a generally rectangular shape dimensioned to slide within a second receiver member 922b of the third plate member 902. The configuration of the slider and receiver members 922a, 922b, 924a and 924b for the plate 900 are not intended to limited to the embodiment described above, but may also include other configurations. The slider and receiver members 922a, 922b, 924a and 924b are intended to be interchangeable, for example, the third plate member 902 may have two receiver members or two slider members which may slidingly couple to a corresponding slider or receiver member on the first and second plate members 904 and 906.

The geometry, features and functions of the slider and receiver members 922a, 922b, 924a and 924b may be the same or similar to the various embodiments described for the plate 100. An additional benefit of the plate 900 is that two sets of slider and receiver members 922a, 922b, 924a and 924b may operate independently of one another. For example, FIG. 12A shows that a distance D1 between the first plate member 904 and the third plate member 902 may be independent of a distance D2 between the third plate member 902 and the second plate member 906. The independence of the first, second and third plate members 904, 906 and 902 may be desirable because the forces acting on vertebrae (or other boney structures) are often different. Since the force acting on the various vertebral levels may differ, a surgeon may want to apply different forces on each vertebrae level using only one plate. The plate 900 may allow for more than one level of vertebrae to be properly stabilized without the need for an additional plate. Although the plate 900 is shown as being adapted to couple to three vertebrae, additional plate members may be added to stabilize more than three vertebrae levels.

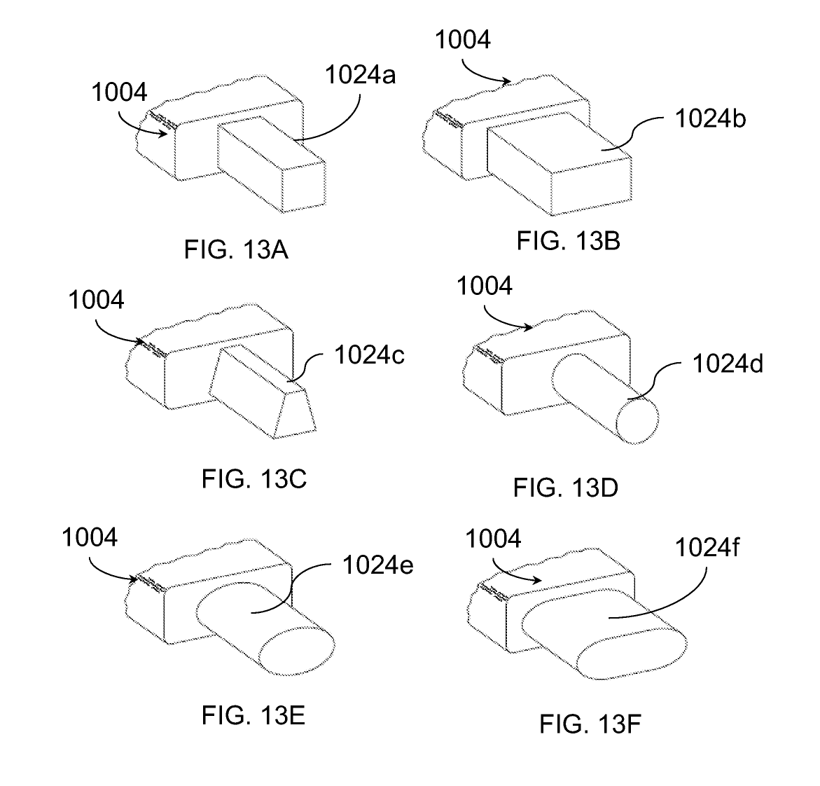

Referring now to FIG. 13A-13F, a second plate member 1004 is presented illustrating alternative embodiments a slider member 1024 which may be incorporated into any of the embodiments disclosed. A wide variety of geometries may be used for the slider member 1024, for example, the second plate member 1004 may have a square cross sectional slider member 1024a, a rectangular cross sectional sliding member 1024b, or a trapezoidal cross sectional sliding member 1024c. Alternatively, the slider member 1024 may have a circular 1024d, oval 1024e or racetrack 1024f geometry. A longitudinal portal of a receiver member (not shown) may have a geometry which corresponds with the shape of sliding member 1024 which may allow the receiver member and the slider member to slide in relation to each other.

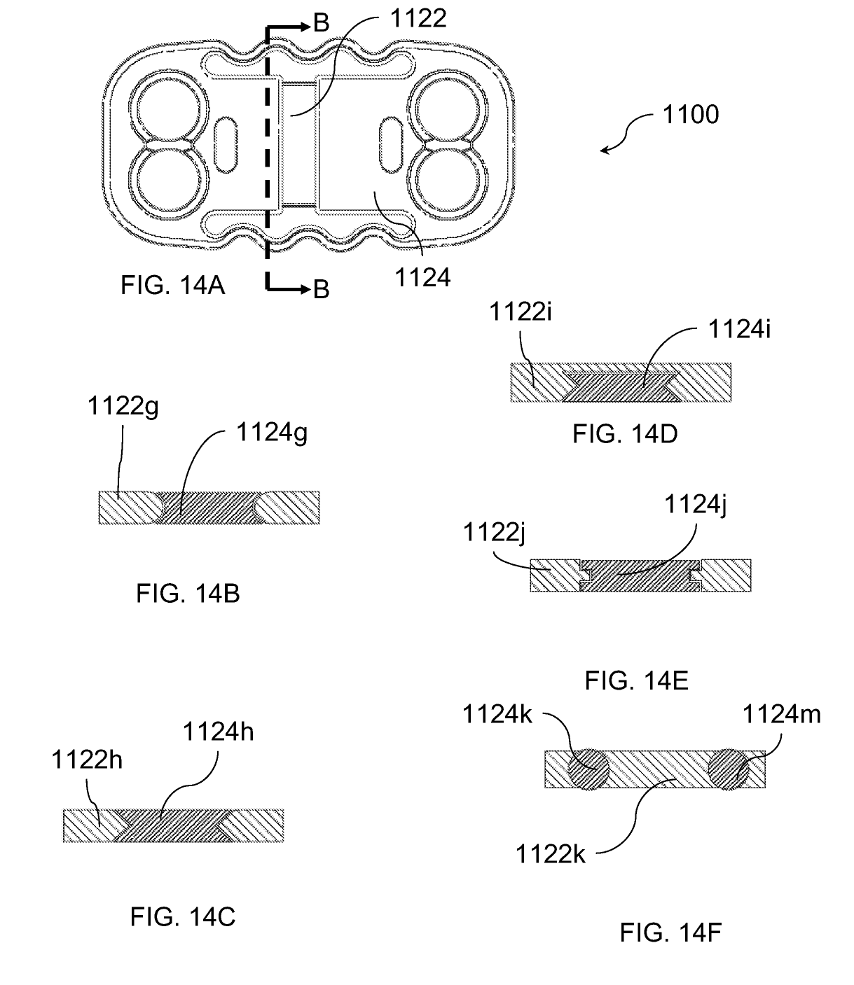

Referring now to FIGS. 14A-14F a detailed cross sectional view of a plate 1100 is presented illustrating additional embodiments of a receiver 1122 and slider members 1124 that may be incorporated in any of the embodiments of the present disclosure. FIGS. 14B-14F show a cross sectional view of a receiver member 1122 and slider member 1124 taken along line B-B of the plate 1100. For example, a slider member 1124g may have a generally rectangular shape with concave side walls which may ride against convex side walls of a corresponding receiver member 1122g as shown in FIG. 14B. Rather than concave side walls, a slider member 1024h may have a triangular shaped side walls that may ride along corresponding angled side walls of a corresponding receiver member 1122h as shown in FIG. 14C. Another possible embodiment may include a tongue and groove slider and receiver members 1124j and 1122j, as shown is FIG. 14E. A pair of tongue members may be located on a receiver member 1122j (as shown) or on the slider member. A pair of corresponding grooves may be located on the slider member 1124j (as shown) or on the receiver member. Other tongue and groove-type configurations are also possible, such as a dove tail designs. The slider member 1124 does not need to be a single member, but may include multiple members as shown in FIG. 14F. FIG. 14F illustrates two circular cross sectional slider members 1124k and 1124m that slide within the corresponding geometry of receiving member 1122k.

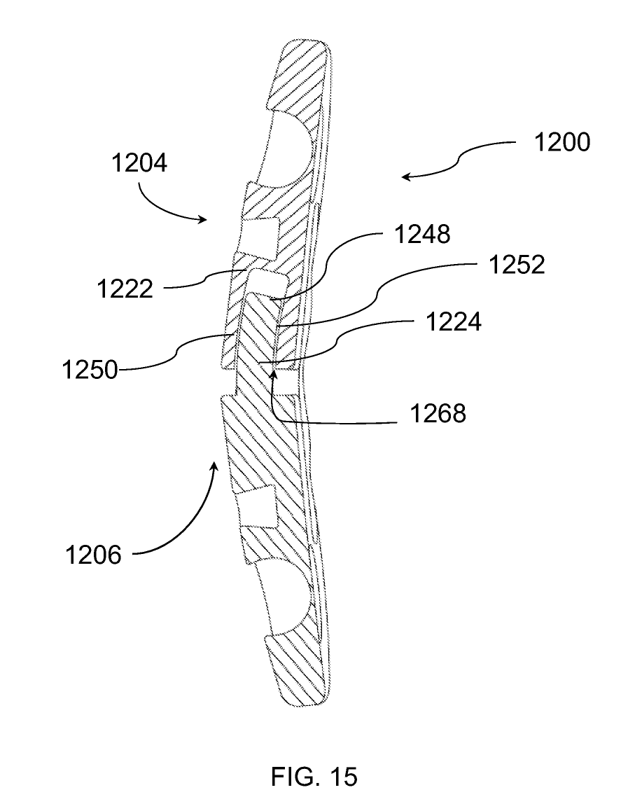

Referring now to FIG. 15 a cross sectional side view of another possible embodiment of a plate 1200 is shown. In addition to the various cross sectional geometries described above, in certain embodiments the plate 1200 may be similar to plate 100 except the plate 1200 may include a first plate member 1204 that includes a receiver member 1222 and a second plate member 1206 that includes a slider member 1224 which follow a curved path. The curved slider member 1224 may have a generally rectangular or race track cross section (although other geometries, such as those described within the current disclosure are possible) which extends axially along a curved path from a second plate member 1206. The curved slider member 1224 may have a top wall 1248, a concave anterior (front) wall 1250, a convex posterior (rear) wall 1252, and two side walls (not shown). The receiver member 1222 may be dimensioned to receive sliding member 1224 as to allow for a gap 1268 between the slider member 1224 and the receiver member 1222. The gap 1268 may be small to minimize any side to side movement of the first plate member 1204 relative to the second plate member 1206. Alternatively, the gap 1268 may be larger to allow pivoting of the first plate member 1204 relative to the second plate member 1206. In certain embodiments, the receiver and slider members shown in FIGS. 13A-13F and FIGS. 14A-14F may also extend along a curved path.

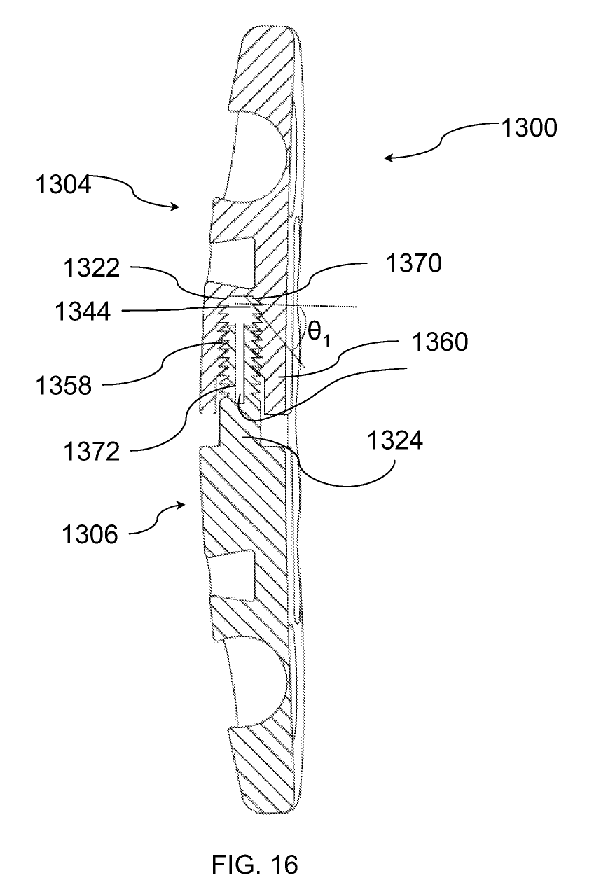

Referring now to FIG. 16, one possible embodiment of a plate 1300 having a ratcheting mechanism that may be incorporated into a first and second plate members 1304 and 1306 is shown. In certain embodiments it may be desirable to the first plate member 1304 and the second plate member 1306 compress and lock together gradually either prior to insertion or after insertion of the plate 1300, such as during settling. Such a mechanism may allow plate 1300 to control the amount of force acting on adjacent vertebrae (or other boney structures). In certain embodiments the first plate member 1304 may include a receiver member 1322 having an anterior (front) wall 1358 and a posterior (rear) wall 1360. The anterior wall 1358 and the posterior wall 1360 may have a plurality of grooves 1370 having an angle θ₁. The grooves 1370 may receive corresponding teeth 1372 on a slider member 1310 which may allow the slider and receiver members 1324 and 1322 to move axially in one direction and while resisting movement in and opposite direction. A longitudinal slot 1380 may extend partially along a center axis of the slider member 1324. The slot 1380 may extend through the slider member 1324 which may allow for deflection of the sliding member 1324 during ratcheting. The slot 1380 may vary in size and shape depending on the desired force required for deflection during ratcheting. The first and second plate members 1304 and 1306 may be compressed together after attachment to the vertebrae so the plate transfers a certain force to the vertebrae being stabilized. After insertion and attachment to the vertebrae the ratcheting teeth features 1370 and 1372 may allow for further compression of the vertebrae as the vertebrae settle.

Although groove features 1370 teeth 1372 are shown as being located on the anterior 1358 and posterior 1360 walls, one skilled in the art would appreciate the groove and the teeth features may be located on the side walls of the sliding member 1310 and slot 1344. One skilled in the art would appreciate that other ratcheting mechanisms that are well known in the art may also be incorporated into plate 700 such as ratchet wheel and pawl combination.

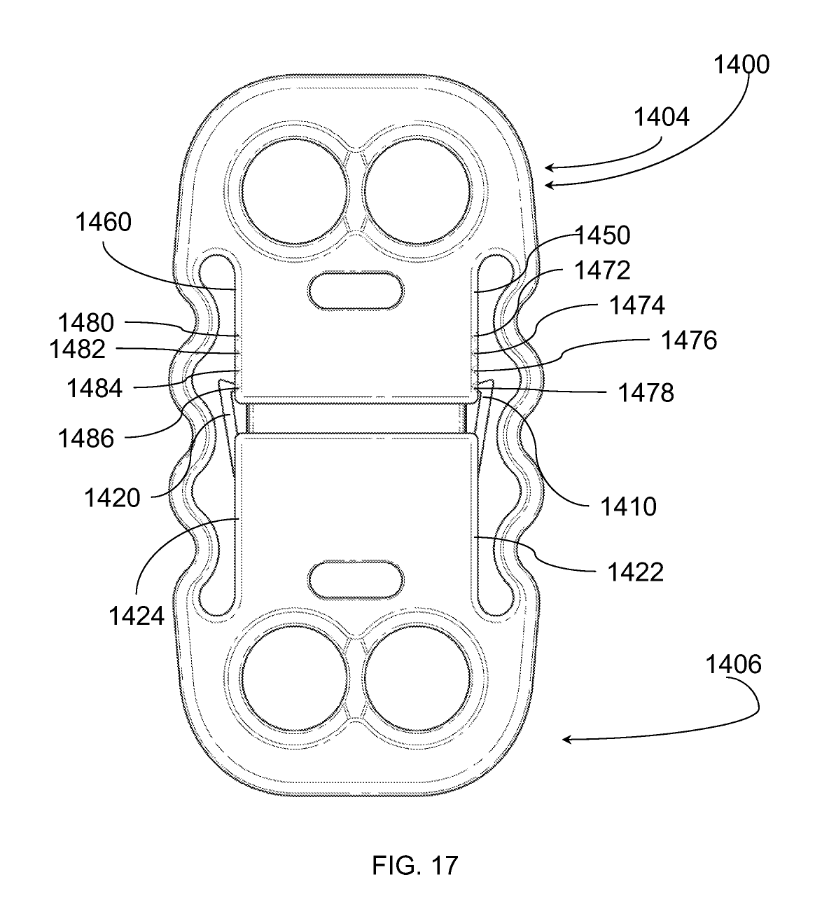

Referring to FIG. 17, a front view of an alternative embodiment of a plate 1400 is presented illustrating another possible embodiment of a ratcheting mechanism. The ratcheting mechanism may control the force applied to a boney structure (such as a pair of vertebrae) and may also be used to aid a surgeon in determining in a non invasive manner the distance the plate 1400 has settled or the force acting on the plate in situ. In certain embodiments the plate 1400 may be similar to the plate 100 as previously described in FIG. 1. The plate 1400 may have a first and second plate members 1404 and 1406. The second plate member 1406 may have one or more one or more side walls 1422 and 1424 each having an opposing indicator arm 1410 and 1420. The first plate member 1404 may have a first side wall 1450 with a series of indicator recesses 1472, 1474, 1476 and 1478 that are dimensioned to receive a portion of the indicator arm 1410. The second plate member 1406 may have a second side wall 1460 with a series of corresponding indicator recesses 1480, 1482, 1484 and 1486 that are dimensioned to receive a portion of the indicator arm 1420. The indicator recesses 1472, 1474, 1476 and 1478, 1480, 1482, 1484 and 1486 may extend partially into the side walls 1450 and 1460. In other embodiments the indicator recesses 1472, 1474, 1476 and 1478, 1480, 1482, 1484 and 1446 may extend through an anterior (front) and posterior (rear) walls of first plate member 1404. The recesses 1472, 1474, 1476 and 1478, 1480, 1482, 1484 and 1486 may represent a distance and/or a force required to move the first plate member 1450 relative to the second plate member 1406.

In certain embodiments the indicator arms 1410 and 1420 may act as a cantilever beam in which a first end of each arm is fixed to the second plate member 1406 and an opposite end of each arm is free and contacts the side walls 1450 and 1460 of the first plate member 1404. The cantilever configuration may allow indicator arms 1410 and 1420 to flex and ride against side walls 1450 and 1460 of the first plate member 1404. The plate 1400 may be expanded prior to or during implantation similar to the plate 100 as previously described. As the first plate member 1404 moves relative to the second plate member 1406, the first ends of indicator arms 1410 and 1420 may slide into and temporarily lock with the first pair of recesses 1578 and 1586. As more force is applied to plate 1400 (either during or after implantation), the end of indicator arms 1410 and 1420 may slide into and temporarily lock with the second pair of recesses 1576 and 1584. As the first plate member 1404 moves closer to the second plate member 1406, the indicator arms 1410 and 1420 may continue to slide and temporally lock with the next pair of indicator recesses 1574 and 1582. Indicator arms 1410 and 1420 may continue to ride and lock into respective indicator recesses until arms 1410 and 1420 slide into the last pair of indicator recesses 1472 and 1480. In certain embodiments the arms 1410 and 1420 may permanently lock into the last pair of indicator recesses 1572 and 1580 to prevent any further movement of the first plate member 1404 relative to the second plate member 1406. The shape and geometry of the indicator recesses 1472, 1474, 1476 and 1478, 1480, 1482, 1484 and 1486 may vary depending on the amount of desired force to move the arms 1410 and 1420 as the plate 1400 settles or compresses.

Other embodiments for a surgical bone plate may include:

1. A bone plate system comprising

a first plate member having a proximal end portion having a plurality of bone screw holes and distal end portion having a longitudinal channel,

a second plate member having a distal end portion having a plurality of bone screw holes, and a proximal end portion that extends along a first longitudinal axis and is positioned at least partially within the longitudinal channel,

a first series of radio opaque markers positioned within the first plate member and aligned along a second longitudinal axis

a second series of radio opaque markers positioned within the second plate member and aligned along a third longitudinal axis that is laterally spaced apart from the second longitudinal axis.

2. The bone plate system of claim 1 wherein the first and second plate members are composed of a radiolucent material.

3. The bone plate system of claim 2 wherein the first and second plate members are composed of a polymer.

4. The bone plate system of claim 1 further comprising a one way ratcheting mechanism within the longitudinal channel

5. The bone plate system of claim 1 further comprising a one way ratcheting mechanism positioned outside the longitudinal channel.

Still other embodiments for a surgical bone plate may include:

1. A bone plate system comprising:

a first plate member composed of a radio opaque material and having a proximal end portion with a plurality of bone screw holes and distal end portion having a longitudinal channel,

a second plate member composed of a radio opaque material and having a distal end portion having a plurality of bone screw holes, and a proximal end portion that extends along a first longitudinal axis and is positioned at least partially within the longitudinal channel,

a first series of radio translucent markers positioned within the first plate member and aligned along a first longitudinal axis

a second series of radio translucent markers positioned within the second plate member and aligned along a second longitudinal axis.

2. The bone plate system of claim 1 wherein first series of radio translucent markers include a first series of apertures extending into the first plate member.

3. The bone plate system of claim 2 wherein second series of radio translucent markers include a second series of apertures extending into the second plate member.

4. The bone plate system of claim 2 wherein a radio translucent material is inserted into the first series of apertures.

5. The bone plate system of claim 4 wherein a radio translucent material is inserted into the second series of apertures.

6. The bone plate system of claim 1 wherein the first longitudinal axis is aligned with the second longitudinal axis.

7. The bone plate system of claim 1 wherein the first longitudinal axis is laterally spaced apart from the second longitudinal axis.

Still other embodiments for a surgical bone plate may include:

1. A bone plate system comprising

a first plate member composed of a radio opaque material and having a proximal end portion having a plurality of bone screw holes and distal end portion having a longitudinal channel,

a one way ratcheting mechanism positioned within the longitudinal channel,

a second plate member composed of a radio opaque material and having a distal end portion having a plurality of bone screw holes, and a proximal end portion that extends along a first longitudinal axis and is positioned at least partially within the longitudinal channel,

a first series of radio translucent markers positioned within the first plate member and aligned along a second longitudinal axis, and

a second series of radio translucent markers positioned within the second plate member and aligned along a third longitudinal axis that is laterally spaced apart from the second longitudinal axis.

Still other embodiments for a surgical bone plate may include:

1. A bone plate system comprising

a first plate member composed of a radio opaque material and having a proximal end portion having a plurality of bone screw holes and distal end portion having a longitudinal channel,

a one way ratcheting mechanism positioned within the longitudinal channel,

a second plate member composed of a radio translucent material and having a distal end portion having a plurality of bone screw holes, and a proximal end portion that extends along a first longitudinal axis and is positioned at least partially within the longitudinal channel,

a first series of radio translucent markers positioned within the first plate member and aligned along a second longitudinal axis, and

a second series of radio opaque markers positioned within the second plate member and aligned along a third longitudinal axis that is laterally spaced apart from the second longitudinal axis.

Still other embodiments for a surgical bone plate may include:

1. A bone plate comprising:

a first plate member extending along a longitudinal axis, having a plurality of bone screw holes and a first receiving segment with a first longitudinal portal;

a second plate member positioned along the longitudinal axis, having a plurality of bone screw holes, a first end portion having a first slider segment positioned at least partially within the first longitudinal portal of the first plate member and a second end portion having a second receiving segment with a second longitudinal portal;

a third plate member positioned along the longitudinal axis, having a plurality of bone screw holes and a second slider segment positioned at least partially within the second longitudinal portal of the second plate member;

a first and second compression biasing members coupled to the first and second plate members and extending generally parallel to the longitudinal axis, wherein the first compression biasing member and the first and second plate members form a continuous wall defining a first window and the second compression biasing member and the first and second plate members form a continuous wall defining a second window; and

a third and fourth compression biasing members coupled to the first and second plate members and extending generally parallel to the longitudinal axis, wherein the third compression biasing member and the second and third plate members form a continuous wall defining a third window and the fourth compression biasing member and the second and third plate members form a continuous wall defining a fourth window.

2. The bone plate of claim 1 wherein the first and second compression biasing members are integral with the first and second plate members so as to form a unitary member.

3. The bone plate of claim 1 wherein the third and fourth compression biasing members are integral with the second and third plate members so as to form a unitary member.

The foregoing details provided regarding the embodiments of the invention have been presented primarily for the purposes of illustration and description. Many of the features and functions of the embodiments described above are intended to be combined into other working embodiments. The details and drawings are not intended to be exhaustive listing of potential embodiments, nor should they limit the invention to the precise forms disclosed. Many modifications, combinations, and variations are possible in light of the above teachings while still remaining within the subject matter of the invention. For example a surgical bone plate may include a first and second plate members, a first and second compression biasing members, a one way ratcheting mechanism, one or more radio opaque markers and a cover. It is intended that the scope of the invention is only limited by the Claims appended hereto. The abstract is in no way intended to limit the scope of the invention

What is claimed is:

1. A bone plate system for stabilizing boney structures comprising:
a first plate member including a first proximal end portion, a first distal end portion having a longitudinal channel, a first and second side surfaces, a bottom surface and a top surface that defines a first recess and a plurality of bores;
a second plate member having a first and second side surfaces, a top surface that defines a second recess and a plurality of bores, a bottom surface, a second distal end portion and a second proximal end portion that extends along a first longitudinal axis and is positioned at least partially within the longitudinal channel;
a first compression biasing member extending from the first side surface of the first plate member to the first side surface of the second plate member, the first compression biasing member including a plurality of successive waves in which the waves include alternating curved crest and curved trough portions;
a second compression biasing member extending from the second side surface of the first plate member to the second side surface of the second plate member, the second compression biasing member including a plurality of successive waves in which the waves include alternating curve crest and curve trough portions, wherein the first and second compression biasing members are laterally offset from the distal end portion of the first plate and the proximal end portion of the second plate;
a plate holder having a first arm with a first end portion positioned within the first recess of the first plate member and a second arm pivotably coupled to the first arm and having a second end portion positioned within the second recess of the second plate member; and
a plurality of screws at least partially positioned within the bores of the first and second plate members.

2. The bone plate system of claim 1 further comprising a force gauge coupled to the first and second arms of the plate holder.

3. The bone plate system of claim 1 wherein the first and second compression biasing member are integral with the first and second plate members so as to form a unitary member.

4. The plate system of claim 1 wherein the second proximal end portion is curved along a second axis that is generally transverse to the longitudinal axis.

5. The bone plate of claim 1 wherein the first and second plate members are composed of a radio lucent material.

6. The bone plate of claim 5 further comprising at least one radio opaque marker positioned within the first or second plate members.

7. The bone plate system of claim 5 further comprising a radio opaque marker positioned within each the first and second plate members.

8. The bone plate system of claim 1 wherein the first and second plate members include a one way ratcheting mechanism.

9. The bone plate system of claim 1 wherein the first compression biasing member and the first and second plate members form a continuous wall defining a first window and the second compression biasing member and the first and second plate members form a continuous wall defining a second window.

10. The bone plate system of claim 1 wherein the second proximal end portion has a generally rectangular cross section.

11. The bone plate system of claim 1 further comprising a cover positioned at least partially about top surface of both the first and second plate members.

12. A bone plate comprising:
a first plate member extending along a longitudinal axis, having a plurality of bone screw holes and a receiving segment with a longitudinal portal;
a second plate member positioned along the longitudinal axis, having a plurality of bone screw holes and a slider segment dimensioned to fit within the longitudinal portal of the first plate member; and
a first and second compression biasing members extending generally parallel to the longitudinal axis and laterally spaced apart from the receiving segment and the slider segment, wherein the first and second compression biasing members are integral with the first and second plate members so as to form a unitary member.

13. The bone plate of claim 12 wherein the first and second compression biasing members are composed of a memory shape material.

14. The bone plate of claim 12 wherein the plurality of bone screw holes of the first or the second plate members overlap.

15. The bone plate of claim 12 wherein the first and second plate members are composed of a radio lucent material.

16. The bone plate of claim 15 further comprising at least one radio opaque marker positioned within the first and second plate members.

17. The bone plate of claim 12 wherein the first and second compression biasing members include a plurality of successive curved segments in which the curved segments include alternating curved crest and curved trough portions.

18. A bone plate system for stabilizing boney structures comprising:
a first compression biasing member extending along a first longitudinal axis, the first compression biasing member including a plurality of successive waves in which the waves include alternating wave crest and wave trough portions;
a second compression biasing member extending along a second longitudinal axis generally parallel to the first longitudinal axis, the second compression biasing member including a plurality of successive waves in which the waves include alternating wave crest and wave trough portions;
a first plate member coupled to a first proximal end portion of the a first and second compression biasing members;
a second plate member coupled to a distal end portion of the first and second compression biasing members; and
wherein the first compression biasing member and the first and second plate members form a continuous wall defining a first window and the second compression biasing member and the first and second plate members from a continuous wall defining a second window.

19. The bone plate system of claim 18 wherein first plate member includes a projection and the second plate member includes a portal dimensioned to slidingly receive the projection.

20. The bone plate system of claim 19 wherein the projection extends along a curved longitudinal axis that is generally parallel to the first compression biasing member.

21. A method of compressing adjacent boney structures, comprising the steps of:
providing a plate having at least one compression biasing member including a plurality of successive curves in which the curves include alternating crest and trough portions,
increasing the axial distance between adjacent crest portions and increasing the distance between adjacent trough portions,
fastening a first end of the plate to a first boney structure with a first and second anchors, and
fastening a second end of the plate to a second boney structure with a third and fourth anchors.

22. The method of claim 21 further comprising the step of locking the first and second anchors together.

23. The method of claim 22 further comprising locking the third and fourth anchors together.

24. The method of claim 21 further comprising the step of aligning at least two holes on the plate.

25. The method of claim 21 further comprising measuring a tension force of the plate.

26. A method of compressing adjacent boney structures, comprising the steps of:
providing a plate having at least one pair of compression biasing members including a plurality of successive curves in which the curves include alternating crest and trough portions,
increasing the axial distance between the adjacent crest portions and increasing the distance between adjacent trough portions,
fastening a first end of the plate to a first boney structure with a first and second anchors, and
fastening a second end of the plate to a second boney structure with a third and fourth anchors.

27. The method of claim 26 further comprising the step of locking the first and second anchors together.

28. The method of claim 27 further comprising locking the third and fourth anchors together.

29. The method of claim 26 further comprising the step of aligning at least two radio opaque markers on the plate.

30. The method of claim 26 further comprising measuring a tension force of the plate.

31. A method of compressing multiple adjacent boney structures, comprising the steps of:
providing a plate having a first and a second pair of compression biasing members including a plurality of successive curves in which the curves include alternating crest and trough portions,
increasing the axial distance between adjacent crest portions and increasing the distance between adjacent trough portions of the first pair of compression biasing members;
fastening a first end of the plate to a first boney structure with a first and second anchors,
fastening an intermediate section of the plate to a second boney structure with a third and fourth anchors,
increasing the axial distance between adjacent crest portions and increasing the distance between adjacent trough portions of the second pair of compression biasing members;
fastening a second end of the plate to a third boney structure with a fifth and sixth anchors.

32. The method of claim 31 further comprising the step of locking the first and second anchors together.

33. The method of claim 32 further comprising locking the third and fourth anchors together.

34. The method of claim 31 further comprising the step of aligning at least two radio opaque markers on the plate.

35. The method of claim 31 further comprising measuring a tension force of the plate.
